(19) United States
(12) Patent Application Publication
(10) Pub. No.: US 2025/0000381 A1
(43) Pub. Date: Jan. 2, 2025

(54) MICRO-DEVICE FOR MEASURING TISSUE IMPEDANCE

(71) Applicant: MB Device LLC, Louisville, KY (US)

(72) Inventors: Thomas V. Saliga, Tampa, FL (US); Vasiliy Abramov, Louisville, KY (US)

(73) Assignee: MB Device LLC, Louisville, KY (US)

(21) Appl. No.: 18/761,755

(22) Filed: Jul. 2, 2024

Related U.S. Application Data
(63) Continuation-in-part of application No. 17/516,805, filed on Nov. 2, 2021.
Application No. 17/516,805 is a continuation of application No. 17/315,551, filed on May 10, 2021, now abandoned.
Application No. 17/315,551 is a division of application No. 16/285,714, filed on Feb. 26, 2019, now abandoned.

(51) Int. Cl. A61B 5/053; G01N 27/02; G01N 33/483; G11C 27/02

(52) U.S. Cl. CPC A61B 5/053; G01N 27/026; G01N 33/4833; G11C 27/02

(57) ABSTRACT

A micro-device for the measurement of impedance of a biological load by electrical impedance spectroscopy technique using low power direct current to simulate alternating current.

### BACKGROUND OF THE INVENTION

There is a need for Electrical Impedance Spectroscopy (EIS) in a variety of industries. For example, this technique, that employs alternating current, is used to measure two-terminal impedance over a wide frequency range in material and biomedical sciences. With measurement frequencies less than about 100 KHz, the EIS technique often uses a Kelvin type connection to the target material. The Kelvin method normally drives a constant and known sinusoidal current of specified frequency into the target material using one pair of electrodes and then, with a second pair of interposed electrodes, measures the real and imaginary voltage components using multiplicative phase detectors, amplifiers and data acquisition methods. The ratio of these “real” and “imaginary” voltages to the known drive current is the impedance at the drive frequency. The physical equipment to implement EIS is normally quite large as compared to, say, a postage stamp. Further, this equipment requires multiple watts of power during operation. However, new applications for EIS require substantial miniaturization and extremely low power compatible with long term, unattended battery operation.

For example, the medical arena of wearable medical monitors is now becoming popular and, in particular, Electrical Impedance Tomography holds promise for identifying various medical disorders before they are otherwise apparent. This application requires that a plurality of EIS devices be placed on a patient's body and that ideally, the person must be ambulatory and able to function normally. Further, EIS has been shown to be particularly helpful in the prediction of fluid build-up in tissue. These applications require a substantially smaller measurement device which operates with micro-watts of average power. No such device is known to be available.

What is sought is an EIS means and method which is capable of measuring biological impedances from 10 Hz to 100 KHz, capable of being miniaturized to approximately one-half the size of a postage stamp with a thickness of tens of mills and able to function on similarly small batteries for days to months. Further, it's collected EIS data must be wirelessly accessed on a regular interval to fulfill its “wearable” mission. The subject invention employs very small micro-computer-based techniques with a minimal number of external components to accomplish the above goals and is hereafter called the Pulsed-Kelvin Impedance Measurement method (PKIM).

### SUMMARY OF THE INVENTION

The system and the method of this invention is herein referred to as Pulsed Kelvin-connected Impedance Measurement (PKIM). PKIM can be employed wherever miniaturization and low power usage are required to determine potential of current flow through a biological load such as, for example, measuring impedance of biological tissue.

In accordance with the invention, timed, binary voltage pulses of known frequency, amplitude and driver resistance are applied to a material, typically a biological load, via at least two separate electrodes operatively connected to specifically timed sample and hold circuits. The voltage output from these sample and hold circuits is converted to numbers with one or more analog-to-digital converters (ADC). By a combination of local data processing and post-processing of these numbers, the desired EIS data is extracted and reported.

### DESCRIPTION OF THE DRAWINGS

FIG. 1 is a schematic representation illustrating the basic elements of a low parts-count and low power circuit connected to an electrical model of the electrodes and tissue in accordance with the present invention.

FIG. 2A illustrates potential input to a biological load showing positive and negative input pulses of selected frequency.

FIG. 2B illustrates preferred pulse sampling points; and

FIG. 2C illustrates a simplified diagram illustrating potential increase from each pulse sampling at output of the sample and hold devices;

FIG. 3A is an electrical schematic of the approximate equivalent circuit of internal human tissue, where Ze2 represents tissue impedance, and is representative of the circuit resistances to be measured;

FIG. 3B is a plot representative of the magnitude of tissue impedance versus frequency;

FIG. 4A is representative of the pulse potential waveform applied across electrodes connected to the tissue to be measured;

FIG. 4B plots the relative voltage of the pulse waveform shown in FIG. 4A;

FIGS. 5A, 5B and 5C detail how a repetitive sampling of a high impedance voltage source may be used to charge a suitably large sample and hold capacitor to enable a slow, low input impedance ADC to be employed in the PKIM measurement process;

FIG. 6 is a logic flow diagram detailing a method to select a suitable pulse generator source resistance at each frequency to be measured;

FIG. 7 is a summary of the formulae used to extract the real and imaginary impedance components of the tissue impedance;

FIG. 8 is a functional block diagram of a preferred embodiment of the invention;

FIG. 9 illustrates a physical overview of one embodiment of the invention; and

FIG. 10 illustrates one application of the embodiment of FIG. 9.

### DESCRIPTION OF THE INVENTION

As used hereinafter the term “biological load” refers to biological tissue for which determination of tissue impedance is desired.

As used herein the term “sample” means a rapid closure, then opening of a switch which is coupled to a time varying voltage source. The switch closure time is understood to be small compared to the time rate of change of the voltage source. This switch may be coupled to an ADC or a sample and hold capacitor for charge-up purposes.

“Sampling” may be considered to be the act of sensing a potential at a specific point in time on a time varying potential source, and this potential source may have a non-zero source impedance.

“Binary” as applied to current pulses means that the pulse may be positive or negative depending on the polarity of the circuit.

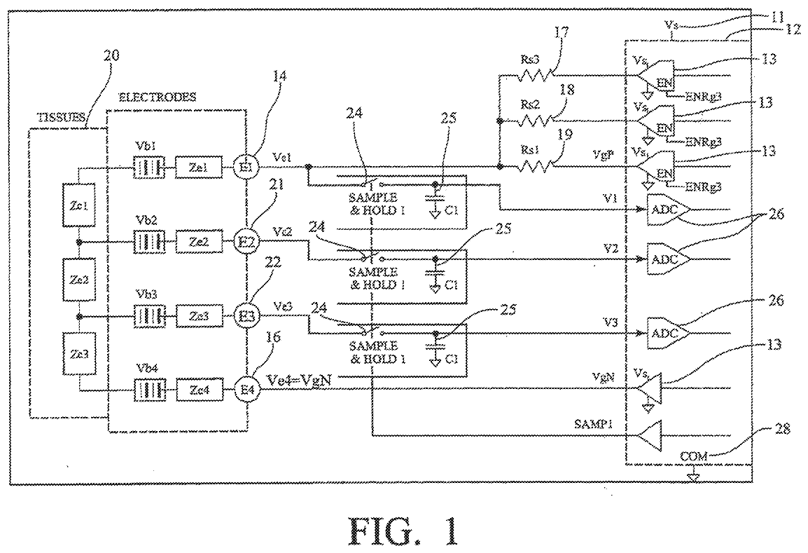

Referring to FIG. 1, the essential hardware elements of the PKIM micro-device 10 are shown along with an electrical model of the electrodes and connected tissues. A primary goal is the measurement of tissue impedance, Ze2, over a range of frequencies as if the binary excitation potentials (VgP and VgN) were pure sinusoidal waves.

As illustrated four-electrode sensing technique, also known as Kelvin sensing, of the micro device of the invention is shown and described. This technique is an impedance measuring technique that employs current-carrying driving electrodes and voltage measuring sensing electrodes. The driving electrodes and sensing electrodes are in separate circuits. It will be understood that Kelvin sensing may be achieved with three electrodes. Best results in regard to component parts, impedance results and miniaturization are obtained by the preferred four electrode technique.

As shown, a low power direct current supply line 11 provides supply current (Vs) from a source of low power direct current (not shown) to a micro-controller 12. Micro-controller 12 controllably drives current-limited current pulses of supply current through resistors 17, 18 and 19 into a biological load via the Kelvin connected positive driving electrode 14 and the Kelvin connected negative driving electrode 16. The current pulses are positive when supply current is drawn through current supply line 11 and negative when supply current is drawn through common line 28. Micro-controller 12 controllably switches between the supply line 11 and the common line 28 to alternate the polarity of the supply current and the resultant pulses.

The current is limited by resistors 17 (1.0K), 18 (3.16K) and 19 (10K) which may be enabled by the micro-controller 12 in any combination. These resistors set a desired range of current injected into the tissues via electrodes 14 and 16. It is understood by one skilled in the art that additional resistors and micro-controller port drivers could be employed if so desired. The actual current driven into the tissues may be computed by measurement of the potential at electrode 14 (Ve1) and a knowledge of the pulse drive voltage, (VgP) for positive pulses and at electrode 16 and a knowledge of pulse drive voltage (VgN) for negative pulses. VgP and VgN are typically either the micro-computer's supply voltage for positive pulse production or supply voltage through the local common 28 for negative pulse production.

The center pair of Kelvin connected sensing electrodes 21 and 22 are each series connected to the fast analog switches 24 which in turn are connected to relatively large storage sample and hold capacitors 25, (C1), and the micro-controller 12 to accumulate charge from specifically timed samples of the tissue potentials through line 38a, 38b and 38c. Micro-controller 12 operates the fast-acting analog switches 24 for timed, rapid closing and opening to make and break a circuit between each of the electrodes 21 and 22 and their respective sample and hold capacitors 25 for sensing a segment of the potential of a pulse at a specific point in time on a pulse waveform and accumulating an incremental segment of the potential of each of the pulses through the bio load until maximum potential is accumulated. The sample and hold capacitors 25 permit conventional, slower sampling analog to digital converters 26 (ADC's) to be used without incorporating active operational amplifier buffers at the electrode nodes 14, 16, 20 and 22. This reduces parts count and power draw, both critical to the intended applications.

As illustrated, the polarity of the circuit is positive, and the resulting pulses are positive. Reversing current flow by drawing the supply voltage through the local common 28 reverses polarity of the circuit and the resulting pulses are negative. Circuit polarity is controlled by the micro-controller 12.

By application of PKIM, applicant:
a. substantially reduces the parts count and size of the circuitry as compared to conventional AC coupled, sine-wave driven, dual-phase detector EIS;
b. measure real and imaginary impedance components of any impedance in a frequency range under approximately 100 KHz provided the impedance does not rise sharply with frequency.
c. in the special case of human tissue impedance, extract the real-component of a low frequency (25 Hz), mid-frequency (2 KHz) and high frequency (100 KHz) equivalent tissue impedance, Re [Zc2], with good accuracy. For example, these particular impedances are an early measure of the physiological condition of tissue;
d. measure the total electrode to electrode impedance (14 to 16) over a similar range of frequencies. For example, in the case of breast tissue impedance monitoring may be a reliable indicator of lymphedema in a continuously monitored patient or as a monitor of pregnancy; and produce a circuit that operates on low power and can be fitted into a small sensing device.

The system of the present invention includes circuitry that is miniaturized and operates on a low voltage DC supply such as a 3 volt battery. Present day micro-controllers such as the Texas Instruments MSP430 series have become quite competent with peripheral resources and the invention's methods take advantage of those capabilities to reduce parts count and size of the PKIM circuit.

Conventionally, an EIS measurement system would use sinusoidal voltage drive signals to the measured tissues. PKIM uses two methods to approximate EIS using a binary pulse driver. The first method modifies the timing on the pulse wave form to better approximate the harmonic content of a sine wave.

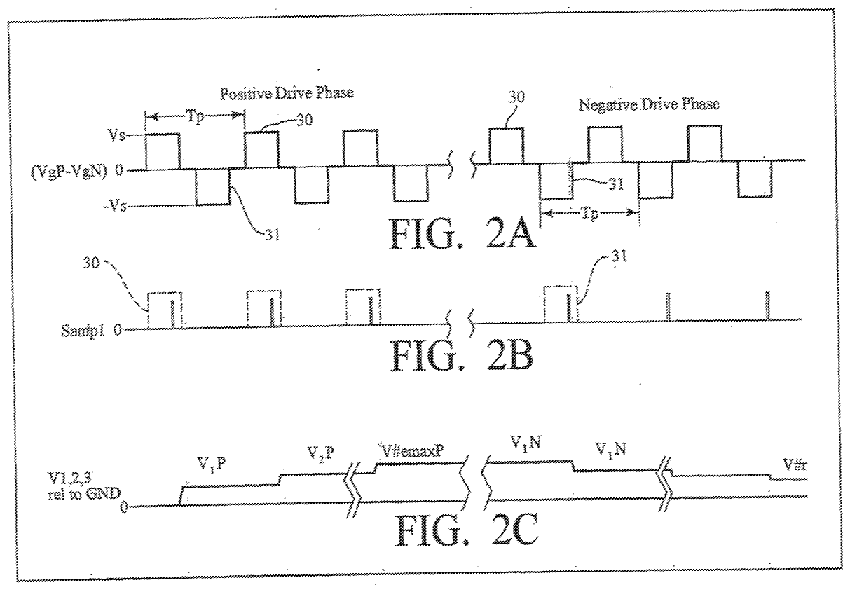

For example, instead of complex sine-wave shaping of the voltage drive to the Kelvin connected electrodes, the micro-controller 12 port-driven, positive pulse (VgP) 30 and negative pulse (VgN) 31 are square wave form as shown in FIG. 2A. These pulses alternate between the known power supply battery voltage and local common 22. For purposes of illustration only three positive and three negative pulses 24 are shown in FIG. 2A. However, it will be understood that the number of pulses, the duration of each pulse, the time of sampling and the number of samples taken is largely dependent on the frequency of the pulses. The micro-controller 12 is programed to select the sampling time and the number of pulses applied across the biological load. One pulse time cycle is shown as Tp.

By proper timing of pulse sampling, the odd harmonic components of these pulses are substantially reduced, permitting a much closer approximation to a sinusoidal drive waveform. As illustrated in FIG. 2B, sample points 30′ and 31′ are chosen at the approximate 135° of the pulse 30 and 31 wave form so that the results more closely emulate a pure sinusoidally driven bio-load and minimize pulse frequency harmonic impedance measurement errors.

FIG. 2C shows the increase in the charge at a sample and hold capacitor 25 as subsequent pulses are sampled. As illustrated, maximum potential is achieved after only three pulses are sampled, however, it will be understood that, due to the rapid closing and opening of the fast-acting switches 24, a larger number of pulses must be sampled to achieve maximum potential. The number of pulses sampled is not critical so long as an enough are sampled to reach maximum potential for the biological load being measured for its impedance. Sampling after maximum potential has been reached has no further effect on the charge of the sample and hold capacitor 25.

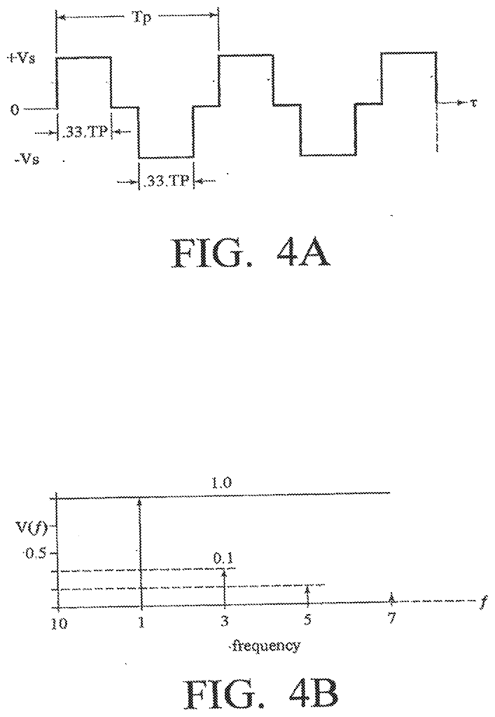

FIG. 4A illustrates the method in which a square waveform is modified such of that the timing of the non-zero potential of a pulse during any ½ the wave form cycle (33Tp) is approximately 66% of the total period (Tp). With this condition, the third harmonic is reduced from ⅓rd, as it would be for a square wave, to approximately one tenth. The higher odd harmonics are also reduced. FIG. 4B plots the relative voltage of the pulse waveform shown in FIG. 4A.

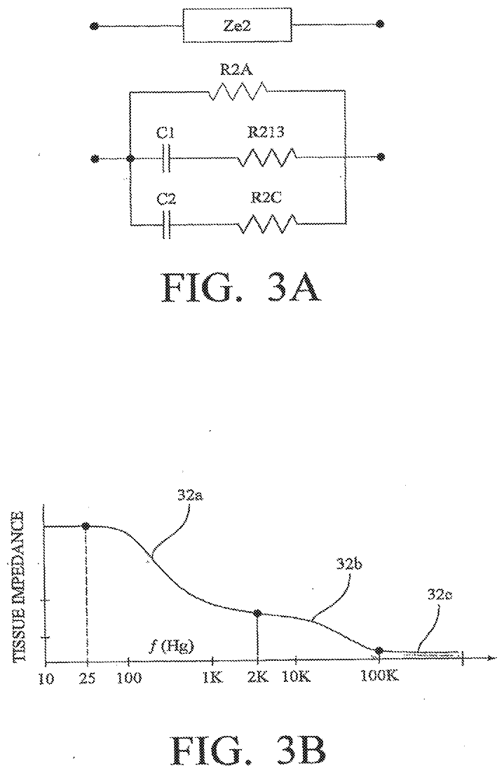

Secondly, in order to approximate a sinusoidal driven EIS system drive frequency is selected in which the pulse harmonics encounter essentially the same impedance as the fundamental frequency. FIG. 3B is a sketch of typical tissue impedance versus frequency. Note that there are three plateaus, 32a, 32b and 32c. By choosing a fundamental frequency at the lowest frequency in each plateau, the pulse's higher harmonics encounter essentially the same resistive value as the fundamental frequency. As illustrated the selection points are located at 25 Hz, 2 KHz and 100 KHz as convenient frequencies. However, other fundamental frequencies can be selected along the plateaus with good results. Thus, by incorporation of the above two steps, the measured EIS impedance closely approximates an ideal sinusoidal based EIS system.

Conventionally, some embodiments of the aforementioned micro-controller 12 include up to four Delta-Sigma, 24 bit Analog-to-Digital convertors (ADC) permitting extra-ordinarily small signals to be digitized with adequate resolution for these EIS applications. However, the Delta-Sigma ADC's acquisition time is too long to acquire phase-sensitive analog data for frequencies much higher than 5 KHz while tissue monitoring may require impedance determination up to 100 KHz. Further, while available micro-controller ADC's incorporate their own sample and hold circuits, these circuits require a low-impedance drive source and are not suitable therefore to be directly connected to biological tissues. PKIM employs a novel repetitive sampling method to affect the desired measurement while using this type of ADC.

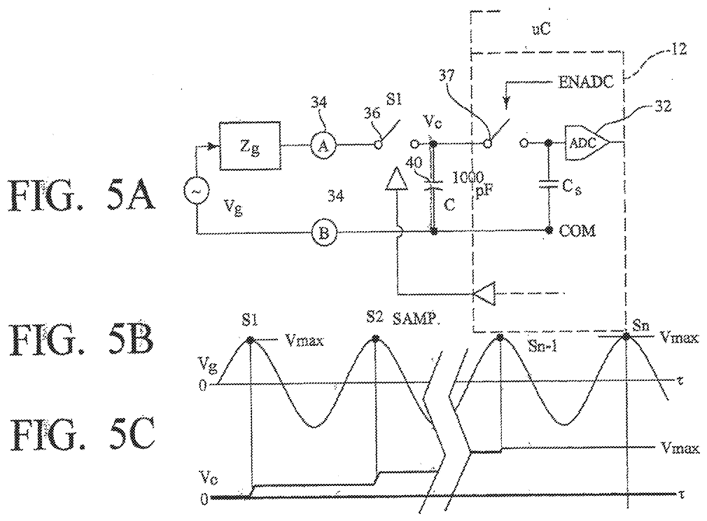

FIG. 5A is a simplified illustration showing a series of positive pulses applied to the biological load until voltage (Vmax) reaches maximum whereupon polarity is reversed and a like series of negative pulses (not shown) is applied until maximum negative voltage (FIG. 5C) is reached in accordance with the circuit diagram to illustrate the principal of PKIM operation. A voltage-generator 34 (Vg) with series impedance, Zg simulates a voltage as might be measured by electrodes connected to tissues. Series impedance, Zg, may have a value in the 5,000-ohm range. PKIM sampling switch 36 rapidly closes and then opens at a desired sample point SI on the Vg waveform as shown in FIG. 5B. Micro-controller 12 controls switch 36 through line 38. The fast-acting switch 24 closure period may be less than 3 microseconds and preferably in the one to two microsecond range. When the switch is closed, the voltage Vg charges the sample and hold capacitor 40, via the series impedance, Zg. The sample and hold capacitor 40 may have a value of approximately 1000 pF. During the short closure time of switch 36, sample and hold capacitor 40 can only charge up partially. It is well known that the Zg x sample and hold capacitor time constant would be about 16 micro-seconds. Thus, a single sample period of two microseconds would not allow the voltage Vc to equal the actual desired voltage, Vmax as shown in FIG. 5B. However, by repeatedly sampling at the same point on the Vg waveform, the sample and hold capacitor 34 will accumulate charge in packets and, with enough samples, the voltage Vc will rise to closely equal Vmax. Once Vmax is reached additional sampling adds nothing the charge of the sample and hold capacitor 40. FIG. 5C illustrates how the voltage across the sample and hold capacitor 40 rises with enough repeated sample cycles to eventually equal the desired sample value. Once the sample and hold capacitor 40 has this final value, no more current is drawn from the tissue source generator and enough energy is stored in the sample and hold capacitor to close switch 37 and complete the circuit to an ADC 32 of a conventional micro-controller 12 so that it can make its conversion with little error. By this means, PKIM eliminates the need for analog buffer amplifiers with a consequent reduction in parts count, size and power draw.

In conventional EIS instrumentation, relatively large AC coupling sample and hold capacitor s are typically used with biological tissue measurements because the electrode to tissue conductors forms a half-cell potential which can severely bias the computed impedance results. This equivalent DC voltage is shown in FIG. 1 as potentials Vb1, Vb2, Vb3 and Vb4. Further, these aforementioned coupling sample and hold capacitor s increase data acquisition time since they may have a significant RC time-constant settling time after a frequency or polarity change. However, the PKIM method employs a novel method to eliminate this requirement which further reduces size, parts count and, due to a lower data acquisition time, it also reduces average power draw.

Electrodes connected to tissues typically develop half-cell potentials as shown in FIG. 1. Each electrode's DC bias voltage, Vb1, Vb2, Vb3 Vb4, may develop DC potentials of 0.2 vdc or more. Unless accounted for or cancelled out, these will cause substantial errors in measurement of the tissue impedance. As noted earlier, typical EIS instruments employ AC coupling sample and hold capacitors.

To forestall their use and keep component count and size to a minimum, PKIM takes advantage of the micro-controller 12 binary port drivers 13 to invert the drive potentials at Ve1 and Ve4 and make a second series of sampled measurements at each frequency so that DC offsets are cancelled. For example, and with reference to FIG. 1 and FIG. 2, as illustrated a first set of sampled measurements (resulting in V1, V2, V3 ADC values) are made with electrode 16 held at common or ground potential so that the binary port drivers 13 act as positive pulse generators to generate the positive pulses VgP that are injected into the biological load 20. Then a second set of sampled measurements are made with electrode 14 held at common while negative pulses VgN produced by the binary port drivers 13 acting as negative pulse generators are injected into the biological load 20. By averaging a “positively driven” impedance and a “negatively driven” impedance, and taking their average, an unbiased impedance value may be derived.

It is important to note that in practice with biological loads, that the electrode DC offsets (Vb #) will change when driven with an AC signal versus direct measurement. Thus, simply measuring Vb # by making VgP and VgN zero and then subtracting that value from AC measured sample values will not usually give a correct result. For instance, in one case, a zero excitation DC potential between two electrodes connected to a biological load measured 55 millivolts but when excited by AC currents, it increased to 83 millivolts. With a 3V supply voltage-based measurement, this difference would cause substantial measurement error if a direct DCV offset correction method had been used.

In a conventional EIS system using Kelvin connected electrodes, a constant-current, sinusoidal signal is applied to the E1 and E4 electrodes. However, due to the low battery voltage (3V approx.) and minimal parts count goal, a constant-current driver is not a good choice. Further, as will be described, the above DC offset correction method requires that the measured voltage drop across the Kelvin connected electrodes (E2, E3) not reverse in potential sign due to comparatively large bias voltages, Vb2, Vb3. This places a lower limit on the current and hence the voltage drop across the biological-load. Therefore, the pulse generator's current sensing resistor, Rg, must not be too large relative to the biological load impedance at the frequency being tested.

There is yet another important PKIM design consideration in the selection of Rg at the frequency being tested. Specifically, the selected Rg and the magnitude of the pulse amplitude potential (i.e.: the supply voltage, Vs) should not cause the total voltage across electrodes E1 and E4 to exceed approximately 2.1 volts or else electrolysis may take place at those electrode sites.

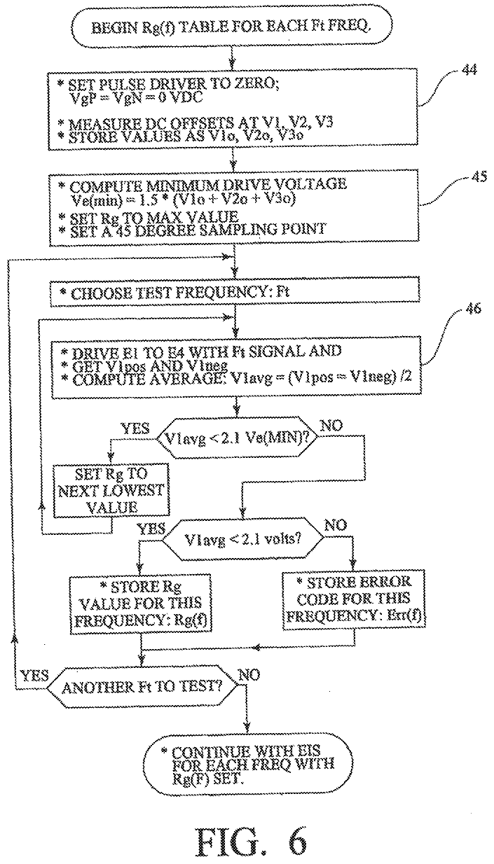

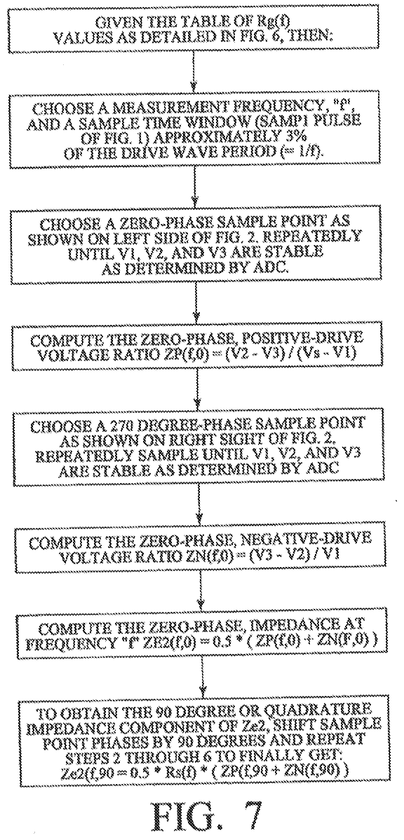

Thus, the choice of resistance, Rg, for proper PKIM operation on biological loads must be “windowed” between some minimum and some maximum value at each frequency measured before the actual measurement is made. A PKIM process for accomplishing this selection automatically is detailed in the flow diagram of FIG. 6. The goal of this Rg selection method is to select a generator driver resistance which assures that the tissue measurement voltages are large enough to overcome the accumulated DC bias offset voltages of the electrodes but not so large as to cause electrolysis to occur. It is assumed that a plurality of Rg selections is implemented.

As shown in FIG. 6, the first actions 44 in the Rg selection process are to measure the zero-drive voltage DC offsets V1o, V2o and V3o as seen at the electrodes 14, 16, 21 and 22. The next step 45 is to establish a minimum peak drive signal value across the biological load which is above the maximum expected accumulated, absolute DC offset value as determined by multiplying the sum of the zero-drive voltage DC offsets from step 45 as seen at the electrodes 14, 16, 20 and 22. In one case, the zero-drive values measured were 40 millivolts, 60 millivolts and 88 millivolts for V1o, V2o, and V3o respectively. Thus, with a 50% safety factor, a minimum electrode drive voltage, Ve (min), was computed to be (1.5×(0.04+0.06+0.088)) 0.282 volts.

The next actions 46 are to iteratively try each Rg value from the highest value toward the lowest values to find a drive potential which first causes the average sampled voltage, Vlavg, to exceed this Ve (min) value. The sampling point was chosen to be at 45 degrees (relative to the drive wave maximum amplitude) to assure approximately equal parts of real and imaginary impedance contributions. Assuming that Vlavg is less than 2.1 vdc, then that Rg value, Rg (f) is stored and used thereafter for all EIS measurements that are made at that frequency. Once Rg values are chosen for each frequency to be measured, the PKIM method then makes impedance measurements at each pre-selected frequency. This Rg selection process is normally only performed once but may be repeated if V1 is determined to be less than Ve (min) at any time in the future.

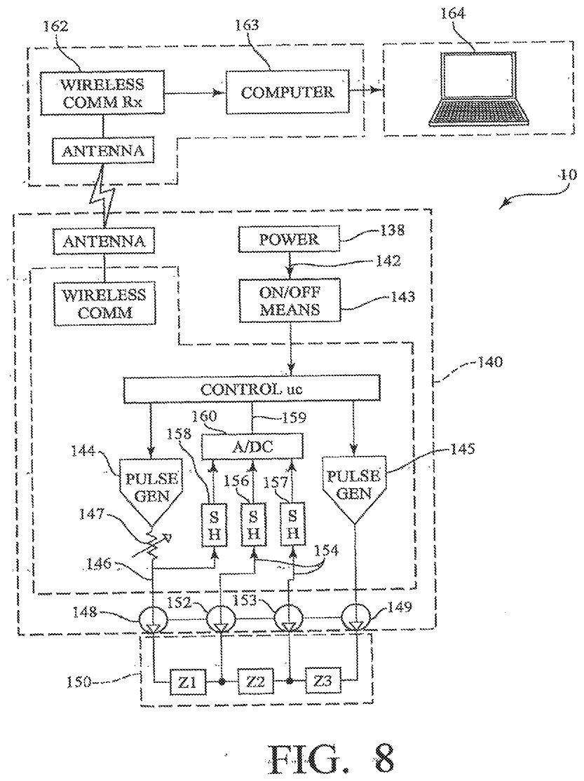

With suitable Rg values chosen, the impedance measurement process may be started. This process is summarized in FIG. 7. The measurement frequency is selected and the Rg for that frequency as determined above and illustrated in FIG. 6 is selected. A series of phase-timed positive samples are taken on each polarity of the drive wave until V1, V2 and V3 are stable indicating that the sample and hold capacitors 23 have been fully charged and the resulting ADC values of V1, and V3 are saved. Polarity is reversed and the ADC values for each frequency and sample phase are uploaded by a wireless link. Then, post processing in a more competent computer is performed. However, FIG. 7 shows the overall computations performed. Note that by taking the average of the ZP and ZN impedances that the electrode DC bias voltages (Vb #) are cancelled out. Thus, AC coupling sample and hold capacitors are not required.

Referring to FIG. 8 a block diagram of one embodiment of the micro-device of the invention is illustrated. In this embodiment power 138 is direct current supplied by a 3V battery to a micro-controller 140 through line 142. An on/off switch 143 in line 142 activates the micro-device. A micro-controller 140 is provided with a pulse generator 144 for positive pulse and a pulse generator 145 for negative pulses. A line 146 connects the pulse generator 144 with an injection electrode 148 for driving positive current pulses into a biological load 150 in which Z1, Z2 and Z3 represent tissue impedance.

For negative current pulses electrode 149 is at common. A variable resister 147 is in the line 146 for controlling current. Electrode 149 drives negative current pulses. The potential of each current pulse is sensed by electrodes 152 and 153 and the sensed charge is passed through lines 154 to sample and hold devices 156 and 157. The sample and hold device 158 determines the potential of the input current pulses before reaching the biological load 150. A series of positive and negative pulses are generated during each sampling cycle. For measuring the positive potential the pulse sampling point is preferably about 90° of the wave form until maximum positive potential is built up at the sample and hold devices 156 and 157. The sampling point along the wave form for the negative pulses is then changed by the micro-controller 140 to about 270° until maximum negative potential is built up at the sample and hold devices 156 and 157. Sampling current pulses after maximum potential is stored by the sample and hold capacitors 156 and 157 has no further effect on the stored charge. This maximum potential is routed through line 159 to an analog to digital converter 160 for digitization and the digitized potential is stored in the random-access memory of the micro-controller 140.

The digitized potential is wirelessly transmitted to an external receiver 162. The external receiver 162 may be a nearby computer for computation to impedance and subsequent display of the computed impedance or a computer or other device, such as a cell phone, that includes a more robust transmission system for relaying the digitized potential to a remote computer 164 for computing the impedance of the positive and negative pulses and for averaging the positive and negative impedance. The averaged impedance results may be displayed on any suitable monitor.

Lymphedema is a chronic disease that produces swelling in body tissue due to fluids caused by a malfunction of the lymphatic system. Breast cancer patients who have had some or all the lymph nodes removed from an arm due to the progress of the cancer are subject to contracting lymphedema. Early detection and treatment can reduce the severity of the disease although in its very early stages (subclinical lymphedema) it is extremely difficult to detect by conventional clinical methods, such as measurement of the dimensions of the arm, to detect swelling in the case of breast cancer patients. Subclinical lymphedema normally progresses into chronic lymphedema. Subclinical lymphedema can exist in the body with no outward or detectable sign for months.

EIS as a screening tool for subclinical lymphedema is under study, particularly as it is non-invasive and is relatively inexpensive. The impedance of the tissue is affected by the buildup of fluid. While EIS is useful for the detection of tissue fluid the procedure requires a source of constant current alternating current and requires components that do not lend themselves to miniaturization. A patient undergoing EIS tissue screening must be physically present at the screening site while impedance data is being collected.

Unlike EIS, the micro-device of the invention employs circuitry that operates on low power DC utilizing fewer and smaller components. The micro-device of the present invention is highly suited for screening purposes as it simulates electro impedance spectroscopy (EIS) in its operation. The circuitry is packaged in a micro-device that can be conveniently worn by the patient even while the patient is ambulatory. With wireless transmission a patient using the micro-device of the invention can engage in normal everyday activities while tissue impedance data is collected at the physician's location.

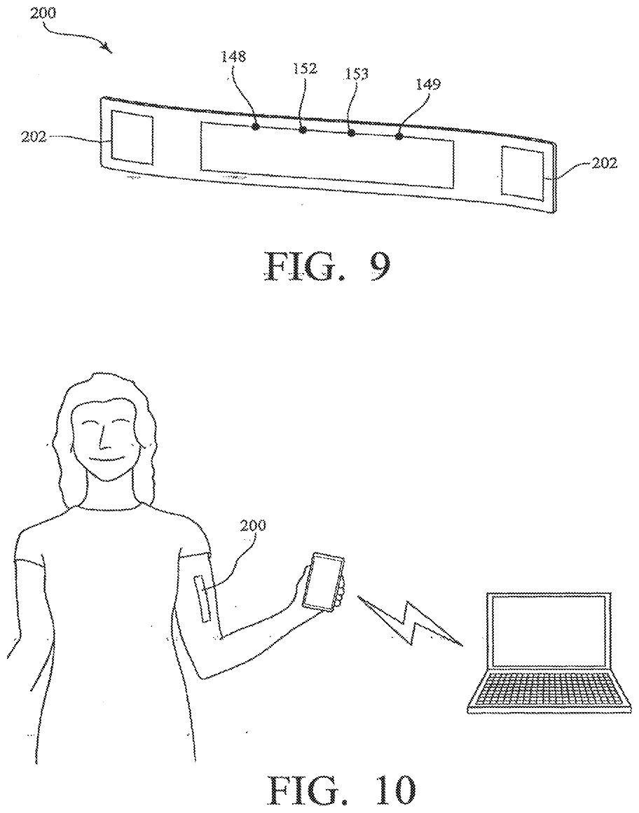

Referring to FIGS. 9 and 10 where like numbers denote like components and like functions, the micro-device 10 of FIG. 8 is affixed to the patient by a strip 200 of surgical tape approximately the size of an adhesive bandage used for minor wounds. An adhesive 202 may be applied at least at both ends of the strip 200 for application of the strip to the area of the patient's body being scanned. Micro-device 10 may be adhesively affixed to the strip 200 or simply secured to the patient by the strip. The electrodes 148, 149, 152 and 153 are exposed for contact with the patient's skin. In the case of a breast cancer patient the strip 200 will normally be placed on the underside of the patient's arm where one or more lymph nodes have been removed during the surgical procedure for removing the cancerous tissue.

As described above in connection with FIG. 8, a series of positive pulses and negative pulses are charged into the tissue of the arm and the resulting positive potential and negative potentials are digitized, averaged and wirelessly transmitted to the external receiver 162. Normally, the external receiver 162 is a cell phone or preferably, a small conventional receiver/transmitter which can be worn by the patient and which automatically transmits the digitized potential to a device, such as a computer, for computation of positive and negative impedance. The positive and negative impedances are averaged to determine the corrected impedance of the patient's tissue.

Good practice will have a micro-device 10 applied on an area of the patient's body, such as the other arm for reference impedance measurements. It will be understood that the strip 200 can be readily moved to other parts of the patient's body such as the leg where lymphedema often occurs. As an alternative embodiment, the micro-device may form part of an elastomeric or adjustable band that can be conveniently worn on the patient's arm or leg.

It will be understood that the micro-device presents a non-invasive and painless method for early detection of tissue fluid and may be utilized in a form that can be worn for an extended period. Persons at risk for tissue fluid build-up, for example breast cancer patients at risk for lymphedema, may be screened immediately after surgery and for a period of time thereafter so that tissue swelling due to fluids may be recognized early on and treatment can be started to reduce the effects of lymphedema.

It will be further understood that the PKIM method described herein can be applied in any situation where simulated alternating current using low DC power is required, particularly where miniaturization is desirable or required.

The present invention allows for the advantages of electro-impedance spectroscopy to be achieved by a micro-device powered by a low voltage direct current battery. The parts count of the micro-device is reduced allowing for substantial miniaturization.

The embodiments disclosed above are illustrative only, as the disclosure may be modified and practiced in different but equivalent manners apparent to those skilled in the art having the benefit of the teachings herein. Furthermore, no limitations are intended to the details of construction or design herein shown, other than as described in the claims below. The embodiments disclosed above may be altered or modified and all such variations are considered within the scope and spirit of the disclosure. Accordingly, the protection sought herein is as set forth in the claims below.

1. A micro-device for the measurement of tissue impedance by electrical impedance spectroscopy technique utilizing low power direct current, said device comprising:
a. a low voltage source of direct supply current,
b. a micro-controller operatively connected to said low voltage source of direct supply current by circuitry comprising a current supply line and a common line, said micro-controller controlling the polarity of said circuitry by alternately switching said direct supply current draw between the current supply line and the common line,
c. a pair of Kelvin connected electrodes comprising a first driving electrode electrically connected to said low voltage direct current supply line and a second driving electrode electrically connected to said common line to complete a current carrying circuit when in contact with a biological load,
d. a pair of Kelvin connected sensing electrodes electrically operatively connected to said micro-controller and at least one fast acting switch by a second independent circuit, said first and second Kelvin connected sensing electrodes disposed between said first and second Kelvin connected driving electrodes for sensing voltage drop through said biological load,
e. said micro-controller controllably generating a series of positive current pulses when said direct supply current is drawn through said current supply line to said biological load through said first driving electrode and controllably generating a series of negative current pulses to said biological load through said second driving electrode when said direct supply current is drawn through said common line,
f. said second independent circuit further including at least one a sample storage and hold capacitor for accumulating microsamples of charge of incremental specifically timed samples of potential of said series of positive pulse signals and said series of negative pulse signals through said biological load as said at least one fast acting analog switch is closed by said micro-controller,
g. at least one analog to digital converter operatively connected to said sample storage and hold capacitor for converting an accumulated charge from said sample storage and hold capacitor to digital format,
h. transmission means for transmitting said accumulated charge to an external receiver for computation of impedance,
whereby alternating current is simulated by injecting a series of positive pulse signals and a series of negative pulse signals of direct current into said biological load and sampling an incremental charge of a micro section of each pulse signal, accumulating said incremental charge of each pulse signal at said sample storage and hold capacitor until maximum potential of said biological load is accumulated, thereafter converting said maximum accumulated potential to digital format and calculating impedance therefrom, the calculated positive impedance and calculated negative impedance is averaged to obtain impedance of the biological load.

2. The micro-device for the measurement of tissue impedance by electrical impedance spectroscopy technique of claim 1, wherein said micro-device circuitry simulates AC coupled, sine wave driven, dual-phase detector electrical impedance spectroscopy using low power direct current.

3. The micro-device for the measurement of tissue impedance by electrical impedance spectroscopy technique of claim 1, wherein said Kelvin connected positive driving electrode drives positive voltage pulses of known frequency into said biological load and said Kelvin connected negative driving electrode drives negative voltage pulses of known frequency into said biological load.

4. The micro-device for the measurement of tissue impedance by electrical impedance spectroscopy technique of claim 1, wherein at least one resistor is provided in said current supply line for limiting supply current frequency.

5. The micro-device for the measurement of tissue impedance by electrical impedance spectroscopy technique of claim 1, wherein said direct supply current is limited by at least three resistors to a low frequency of 25 Hz, a mid-frequency of 2 KHz and a high frequency of 100 KHz, said at least three resistors are electrically connected to said direct current supply line and operatively controlled by said micro-controller for selection of said direct supply current frequency.

6. The micro-device for the measurement of tissue impedance by electrical impedance spectroscopy technique of claim 1, wherein said series positive pulse signals and said series negative pulse signals are square wave form.

7. The micro-device for the measurement of tissue impedance by electrical impedance spectroscopy technique of claim 1, wherein said at least one fast acting analog switch is closed by said micro-controller for a closure period of less than three microseconds to obtain a microsample of potential of each said positive and negative pulse signal.

8. The micro-device for the measurement of tissue impedance by electrical impedance spectroscopy technique of claim 1, wherein said each pulse of said_series of positive and negative pulses is sampled at precisely a same point on a wave form of each said pulse of said series.

9. The micro-device for the measurement of tissue impedance by electrical impedance spectroscopy technique of claim 1, wherein said each said pulse of said series of positive and negative pulses is sampled at precisely the same time during the closure period of said fast acting analog switch.

10. The micro-device for the measurement of tissue impedance by electrical impedance spectroscopy technique of claim 1 wherein said each said pulse of said series of positive and negative pulses is sampled at 66% of said fast acting switch closure period.

11. The micro-device for the measurement of tissue impedance by electrical impedance spectroscopy technique of claim 1, wherein each pulse of said series of positive pulses is sampled at about 90° of said wave form.

12. The micro-device for the measurement of tissue impedance by electrical impedance spectroscopy technique of claim 1, wherein each pulse of said series of negative pulses is sampled at about 270° of said wave form.

13. The micro-device for the measurement of tissue impedance by electrical impedance spectroscopy technique of claim 1, wherein said micro-controller controllably generates positive pulse signals and said Kelvin connected positive driving electrode is operatively connected to said micro-controller for driving said series of positive pulse signals into a biological load and said micro-controller controllably generates negative pulse signals and said Kelvin connected negative driving electrode is operatively connected to said negative pulse generator micro-controller for driving said series of negative pulse signals into said biological load, said micro-controller alternately switching between generation of positive pulse signals and negative pulse signals, said Kelvin connected sensing electrodes are each series connected to said at least one fast analog switch and said sample storage and hold capacitor for accumulating microsamples of charge of incremental specifically timed samples of potential of said series of positive pulse signals and said series of negative pulse signals through said biological load as said fast acting analog switch are closed by said micro-controller.

14. A micro-device for the measurement of tissue impedance by electrical impedance spectroscopy technique utilizing low power direct current comprising:
a. a Kelvin connected first circuit comprising a source of low power direct current, a direct current supply line operatively connected to said source of low power direct current and operatively connected to a first driving electrode, a common line operatively connected to a second driving electrode,
b. a micro-controller electrically connected to said direct current supply line and said common line for driving a series of positive pulses through said first driving electrode when said low power direct current is drawn through said direct current supply line and for driving a series of negative pulses when said low power direct current is drawn through said common line, said micro-controller operatively controlling switching between said direct current supply line and said common line,
c. a kelvin connected second circuit comprising a pair of sensing electrodes, each said sensing electrode series connected to said micro-controller through a fast acting analog switch for sampling an increment of potential of each positive pulse and each negative pulse as said fast acting analog switch is closed by said micro-controller, a sample and hold capacitor for accumulating increments of pulse potential, an analog to digital converter for converting said collected increments to digital format,
whereby alternating current is simulated by injecting a series of positive and negative pulses of direct current into a biological load and sampling an incremental charge of a micro section of each pulse and accumulating said incremental charge of each pulse until positive and negative maximum potential of said biological load is accumulated, thereafter converting said maximum positive and negative accumulated potential to digital format, computing impedance therefrom and averaging the positive and negative impedance to obtain the impedance of the biological load.
